(19) United States
(12) Patent Application Publication
(10) Pub. No.: US 2011/0287534 A1
(43) Pub. Date: Nov. 24, 2011

(54) AUTOMATED FILLING OF FLEXIBLE CRYOGENIC STORAGE BAGS WITH THERAPEUTIC CELLS

(75) Inventors: Jonathan Rowley, Walkersville, MD (US); Patrick Newsom, Middletown, MD (US)

(73) Assignee: LONZA WALKERSVILLE, INC., Walkersville, MD (US)

(21) Appl. No.: 13/099,963

(22) Filed: May 3, 2011

Related U.S. Application Data
(60) Provisional application No. 61/331,201, filed on May 4, 2010.

(51) Int. Cl. C12N 5/071; C12M 3/00

(52) U.S. Cl. 435/374; 435/307.1

(57) ABSTRACT

An apparatus and processes for aseptically dispensing live mammalian cells into sterile, flexible bags in a non-sterile atmosphere. The method includes the steps of: providing the cells suspended in a liquid; providing a plurality of sterile flexible bags fluidly connected to a main line by a plurality of branch lines of sterile flexible tubing; evacuating air from the flexible bags by applying a vacuum to the open end of the main line; preventing fluid flow through all branch lines except that of one bag to be filled; dispensing a desired volume of cell suspension into the open end of the main line; and introducing sufficient sterile purging gas under pressure into open end of the main line to drive into the bag any of the dispensed volume remaining in the main line or branch line of filled bag. Cells can be cryogenically preserved in the filled bags.

### CROSS-REFERENCE TO RELATED APPLICATIONS

This application claims the benefit of priority from U.S. Patent Application Ser. No. 61/331,201 filed May 4, 2010, the contents of which are incorporated herein by reference.

### FIELD OF THE INVENTION

The present invention relates to methods and systems for manufacturing, storing and distributing somatic cell therapy products that comply with regulatory agency requirements, such as current good manufacturing practice (cGMP) regulations for devices, biologics and drugs. More in particular, the present invention relates to partly or wholly automated, closed systems, apparatus and methods for filling containers, particularly flexible cryogenic storage bags, with therapeutic products containing live cells.

### BACKGROUND

The FDA defines cell therapy as the prevention, treatment, cure or mitigation of disease or injuries in humans by the administration of autologous, allogeneic or xenogeneic cells that have been manipulated or altered ex vivo. The goal of cell therapy, overlapping that of regenerative medicine, is to repair, replace or restore damaged tissues or organs.

Ex vivo expansion of cells obtained from human donors is being used, for example, to increase the numbers of stem and progenitor cells available for autologous and allogeneic cell therapy. For instance, multipotent mesenchymal stromal cells (MSCs) are currently exploited in numerous clinical trials to investigate their potential in immune regulation, hematopoiesis, and tissue regeneration. The low frequency of MSCs in tissue necessitates cell expansion to achieve transplantable numbers.

The challenge for any cell therapy is to assure safe and high-quality cell production. In particular, cell processing under current Good Manufacturing Practice (cGMP)-graded conditions is mandatory for the progress of such advanced cell therapies. For allogeneic therapies, the economics of testing and certification of processes and products for GMP compliance are a significant cost factor in cell manufacturing, strongly encouraging production of maximum batch size and minimum batch run.

Optimally, therefore, therapeutic cell manufacturing for clinical-scale expansion would be conducted in a completely automated closed process from cell collection through post-culture processing. Such a closed process would facilitate cGMP-compliant manufacturing of cell therapy products in a form suitable for storage and ready for use in a clinical setting, with minimal risk of microbial contamination and viability losses due to mechanical or physiological stress.

Large-scale automated, closed processes for use of mammalian cells to manufacture proteins, such as biotherapeutics, are well established. However, most such processes are designed to recover a protein product and discard the cells under conditions leading to cell death, either intentionally, as when cells are disrupted to release of intracellular products, or incidentally, when cells are separated from secreted products by harsh methods such as high speed centrifugation. In contrast, processing of therapeutic cells after expansion typically requires cell harvesting, volume reduction, washing, formulation, filling of storage containers and cryopreservation of the product cells, all under conditions maintaining cell viability and, ultimately, clinical functionality.

In addition, therapeutic cells may not survive known processes for handling cells used for protein production because the latter typically represent highly-manipulated cell lines which, during extensive replication in culture, may have undergone selection for less sensitivity to mechanical shear forces and physiological stresses than exhibited, for instance, by progenitor or stem cells used in cell therapies. Thus, to retain efficacy, therapeutic cells typically are minimally cultured so as to maintain the original parental phenotype displayed upon isolation from human tissue; and hence, therapeutic cells generally are not selected or genetically engineered to facilitate downstream processing.

Historically, ex vivo expansion of mammalian cells to obtain increased numbers of functionally useful cells has largely been performed manually, by specialist staff using highly complex manipulations, frequently with an open apparatus. Such special skills are highly individualized, making many such manual techniques difficult to reproduce and even more difficult to scale and convert to automated, closed processes using available cell culture technologies.

Yet, after early stage clinical trials on a proposed cell therapy product have been initiated, there is a substantial incentive to maintain the same basic manufacturing process steps throughout the trials. Thus, process changes made prior to license approval are submitted to the FDA via an amendment of an INDA (Investigational New Drug Application). The degree to which comparability must be established depends upon the scientific basis for predicting any product or process changes, the extent of the process changes, and the stage in product development. The general approach is to establish analytical and biological product comparability by in vitro means. When significant differences are apparent, then it is usual to compare old and new products in animal models of pharmacokinetics, toxicology and efficacy, depending on their availability and relevance. Finally, if differences are apparent in animal studies, it may be necessary to perform clinical equivalence studies in human subjects to establish equivalent safety, pharmacokinetics and efficacy. The nature and extent of such clinical trials required to show clinical comparability for a given product and situation are judged on a case-by-case basis. Additional preclinical or clinical testing is a very costly process and is avoided whenever possible.

The FDA has recognized that it is impractical to lock a process when a product first enters clinical trials and to refrain from making process improvements throughout the product's commercial life cycle for fear of provoking a need to file a new product license application. For certain biotechnology produced products, such as monoclonal antibodies, the FDA has been developing relatively simplified approaches for demonstrating that the products made by the old and new schemes are comparable, through process validation (e.g., virus clearance studies, removal of contaminants or leachables) for all affected process steps. For therapeutic cells, however, the scientific basis for predicting effects of any process change on human efficacy is generally much less established than for therapeutic proteins, due to the higher complexity and fragility of the therapeutic cells and often less availability of in vitro and animal models of efficacy. Hence, as pre-approval clinical trials progress from Phase I to Phase III, there is a substantial economic incentive to meet the steadily increasing demand for therapeutic product by simply scaling up or out the basic manual manufacturing process developed for the earliest human testing.

Accordingly, given the growing number of therapeutic cell products in early stage clinical trials, to minimize inadvertent product changes that require clinical equivalence studies in human subjects, there is a need for cGMP-compliant, automated closed processes that can produce large batches of therapeutic cell products, containing several orders of magnitude more cells than made by largely manual traditional processes, with minimal changes to the basic principles of traditional process steps.

Cryopreservation for long-term storage of animal cells, including therapeutic cells, provides one example of a key downstream cell manufacturing process that has traditionally employed labor-intensive manual techniques. Thus, U.S. Pat. No. 6,136,525 (“the '525 patent”) on a “Method of Cryopreserving Hepatocytes,” issued to Mullon et al., Oct. 24, 2000, discloses cryopreservation of hepatocytes by dispensing them into freezing containers, freezing the containers from between minus 50 to minus 90 degrees Celsius and storing the containers in liquid or vapor nitrogen. See Abstract. The '525 patent further teaches that in the disclosed cryopreservation method, most preferred is a cryoprotectant medium comprising 10% FBS [fetal bovine serum] and 10% DMSO [dimethylsulfoxide]. See col. 3, ln. 5-8. The cells are dispensed into freezer resistant containers, most preferably Cryocyte™ plastic bags from Baxter International, Inc., having a capacity ranging from about 50 to about 500 mL, with syringe-assisted dispensing into such freezer bags being most preferred. See col. 3, ln. 16-34. In this process, cells are funneled through the syringe by gravity or dispensed by gentle pressure into the bags. Once the cells are introduced into the bag, the syringe may also be advantageously used to remove excess air which later promotes the thawing process. The bags are thereafter sealed. Sealing methods may comprise mechanical aluminum seals, thermal impulse heat sealers, luer lock plugs, and the like, with heat sealing being most preferred. Ibid.

The fact that successful cryopreservation and recovery requires removal of air from containers filled with cells in cryoprotectant medium, before freezing, is generally recognized and manually performed. For instance, besides the above disclosure of this point in the '525 patent, U.S. Pat. No. 5,564,279 (“the '279 patent”) on “Freezing Bags,” issued to Thomas et al. on Oct. 15, 1996, discloses a freezing bag for the storage of blood cells and, further, a method of freezing red blood cells using the disclosed bag, comprising, after filling the bag with cells and cryoprotectant through a tube connected to the bag, and before sealing the tube, manipulating the bag to expel, as far as is practical, all air therefrom. See, e.g., claim 11, col. 5, ln. 45-col. 6, ln. 5.

Accordingly, there is a need for improved processes for manufacturing therapeutic cells, from cell collection through post-culture processing, including processes for filling containers with cells suspended in cryoprotectant, particularly such processes that maintain the basic principles of the traditional manual processes yet facilitate manufacturing in automated, closed systems.

U.S. Pat. No. 4,021,283 (“the '283 patent”) on a “Method of Making Aseptic Packaging,” issued to Weikert on May 3, 1977, discloses a process which includes making an aseptic web of bags by first blow-extruding a continuous, closed thermoplastic tube using a non-contaminating gas, dividing the tube by means of partial, transverse heat seals into a series of interconnected bags intercommunicating with each other in a closed system by means of a continuous channel running across their open mouths and then, while maintaining the closed and hence, sterile condition of the web of bags, filling the bags with a sterile product and sealing the bags, to produce sealed, aseptic packages. See Abstract. The '283 patent evidently does not contemplate removal of air or other fluid from the disclosed bags, either before, during or after filling.

U.S. Pat. No. 4,964,261 (“the '261 patent”) on a “Bag Filling Method and Apparatus for Preparing Pharmaceutical Sterile Solutions,” issued to Benn on Oct. 23, 1990, discloses a bag filling method and apparatus for preparing pharmaceutical sterile solutions in a plurality of sterile flexible bags in a non-sterile environment, which method and apparatus comprise providing a pre-sterilized tubular bag having a single inlet, introducing a solution through a sterilizing filter, introducing the sterile solution into the inlet of the tubular bag, and sealing defined sections of the tubular bag after filling to a defined volume of the sterile solution to form a plurality of separate, flexible, sterile bags. See Abstract. The '261 patent evidently does not contemplate removal of air or other fluid from the disclosed bags, either before, during or after filling.

The '261 patent also alleges that U.S. Pat. No. 4,610,790 (“the '790 patent”) on a “Process and System for Producing Sterile Water and Sterile Aqueous Solutions,” issued to Reti et al. on Sep. 9, 1986, discloses a method and apparatus to ensure sterile filling of bags to a high enough level that the bags will be safe for containing intravenous fluids for human use. See '261 patent, col. 1, ln. 20-58. According to the '261 patent, individual bags of sterile solution are produced through using a bag set consisting of 18 individual, flexible vinyl bags attached by means of tubing to a manifold containing 18 valves in turn attached to a sterilizing filter, all pre-sterilized after assembly into a bag set at the factory where the bag set is assembled. Ibid. The '790 patent discloses that: the package for the dilute solution includes a sterile container and tubing which have been sterilized by any conventional means; the container and tubing are formed integrally with a filter housing containing a filter which also are pre-sterilized; the tubing is attached to the filter housing so that the dilute solution produced by the system of this invention can be delivered into the container through the filter which is adapted to retain microorganisms; and the housing can be connected to a plurality of containers, such as flexible transparent bags formed of plastic composition, with appropriate tubing connections so that all water or aqueous solution directed to each bag passes through the filter. See col. 7, ln. 17-40. The '790 patent evidently does not contemplate removal of air or other fluid from the disclosed bags, either before, during or after filling.

U.S. Pat. No. 5,641,004 (“the '004 patent”) on a “Process for Filling a Sealed Receptacle Under Aseptic Conditions,” issued to Py on Jun. 24, 1997, discloses an automated process for filling a sealed receptacle that has at least one part made of a material capable of being pierced by a hollow needle and sufficiently flexible to close itself up again after removal of the hollow needle. See Abstract. In the automated process, the flexible part of the sealed receptacle is pierced using a hollow filling needle which is in contact with the fluid to be channeled into the receptacle. During the process of filling the receptacle, the perforating end of the hollow filling needle is maintained under aseptic conditions by means of laminar gas flow. Ibid. The '004 patent further discloses that the sealed receptacle to be filled can be for example and preferably a bag, filled with gas or on the contrary evacuated of gas, constituted by a flexible material such as an elastomer (see col. 2, ln. 40-42), and that using a hollow evacuation needle of the same type as that used for the filling will allow the fluid already existing in the receptacle to be evacuated. (see col. 4, ln. 27-36). The '004 patent also states that, “[i]f desired, this evacuation can be obtained due to the simple injection of the filling fluid into the receptacle. It may also be preferred to assist this evacuation using for example an evacuation device, preferably in synchronization with the filling, such as a pump. The evacuation could take place before, during or after the filling.” Id.

U.S. Pat. Nos. 6,712,963 (issued on Mar. 30, 2004), and 7,052,603 (issued May 30, 2006), both to Schick, on “Single-Use Manifold for Automated Aseptic Transfer of Solutions in Bioprocessing Applications,” and U.S. Pat. App. Pub. No. 2006/0118472, published Jun. 8, 2006 by Schick et al., on “Single-Use Manifold and Sensors for Automated Aseptic Transfer of Solutions in Bioprocessing Applications,” all disclose presterilized manifolds designed for sterile packaging and single-use approaches. See Abstracts. Disposable tubing and flexible-wall containers (e.g., bags) are assembled via aseptic connectors. These manifolds interact with at least one remotely controlled pinch valve which engages only the outside surface of the manifold tubing. Such manifold and pinch valve systems can be used in conjunction with a peristaltic type of pump, which, together with the remotely operated pinch valve, can be operated by a controller which provides automated and accurate delivery of biotechnology fluid in an aseptic environment while avoiding or reducing cleaning and quality assurance procedures. Each of the collection/storage bags of the disclosed manifolds has three tube connections, including a primary inlet tubing, a second tubing that is used to relieve any gas and/or pressure buildup inside the bag during the filling operation, and an auxiliary inlet/outlet for recirculation of the bag contents.

### SUMMARY OF THE INVENTION

The present invention provides an apparatus and processes for aseptically dispensing live mammalian cells into sterile, flexible bags in a non-sterile atmosphere. The invention is particularly useful for live mammalian cells that are used in a therapeutic product, such as for cryogenic preservation of such cells.

This, one aspect of the present invention provides a method for aseptically dispensing live mammalian cells into sterile, flexible bags in a non-sterile atmosphere, said method comprising the steps of: (a) providing a plurality of sterile flexible bags; (b) providing the live mammalian cells suspended in a liquid contained in a sterile cell source container; (c) connecting the sterile bags to the cell source container, a vacuum source; and a sterile purging gas source to form a sterile system that is closed to the external environment, such that this system selectively allows fluid flow between the sterile bags and either the vacuum source, the cell source container or the sterile gas source; (d) evacuating air from at least one of the flexible bags by selectively allowing fluid flow between at least one bag and said vacuum source; (e) dispensing a desired volume of the liquid for at least one bag by selectively allowing that desired volume flow between that bag and the cell source container; and (f) forcing into the filled bag any of the desired volume of liquid remaining in the system between the cell source container and the filled bag, by selectively allowing sterile purging gas to flow from said gas container through the system to the one bag.

In one embodiment of this method of the invention, the plurality of flexible bags, cell source container, vacuum source and sterile purging gas source are fluidly connected together, at least in part, by flexible tubing. In this embodiment, at least one pinch valve that externally engages the flexible tubing is provided to selectively allow fluid flow between the sterile bags and either the cell source container or the vacuum source or the gas source. In this method it is advantageous to use a peristaltic pump unit to pump the liquid from the source container through the flexible tubing into the flexible bags, so that the entire system remains closed to the outside and no moving pump parts of the pump, which could be difficult to sterilize, contact the liquid inside the sterile system. In some embodiments of this method, either the pump unit or at least one of the pinch valves, or both the pump and one or more valves is remotely operable by a controller.

In a particular embodiment of this method, the flexible tubing in the closed sterile system comprises a main line and at least one branch line. This main line is a length of flexible tubing having one end connected to the cell source container and the gas source so as to selectively allow fluid flow into the main line from that container or the gas source, for instance by a two way valve or by a Y connector and pinch valves. In this embodiment, each of the flexible bags is fluidly connected to the main line by a branch line, with each branch line being a length of flexible tubing having one end fluidly connected to one flexible bag and the other end fluidly connected to the main line so as to allow liquid or gas to flow from main line into the bag. In addition, the vacuum source is fluidly connected to the main line so as to allow that vacuum source to withdraw gas from each of the bags through mainline and the branch lines. The vacuum source can be connected anywhere on the main line, not necessarily close to the connections of the cell source container and gas source, and the bags may be evacuated all at the same time or selectively, by the use of additional pinch valves to selectively close branch lines of different bags.

In some embodiments, the method of the present invention for aseptically dispensing live mammalian cells into sterile, flexible bags further comprises sterilely sealing and disconnecting the branch line of a bag that has been filled, for instance, by heat welding of the tubing.

In the methods of the present invention, viability of cells in the last bag to be filled is at least 90%, 95%, 98%, or 99% of the initial viability of cells in the cell source container before dispensing any of the liquid to fill the bags. For instance, in filled bags at least about 70%, 80%, 90%, 95%, 98%, or 99% of the cells are viable after bag filling. The method maintains high viability when the filling flow rate of liquid through the tubing is from about 10 mL/min to over 1 L/min, the density of cells in suspension is from about 1 to about 30 million/mL, and the liquid comprises from 0 to about 10% dimethylsulfoxide (DMSO) which is used as a cryopreservative as well known in the art. Another aspect of the invention, therefore, is a method of storing live mammalian cells comprising dispensing the cells into sterile, flexible bags according to the invention method for aseptically dispensing live mammalian cells into sterile, flexible bags and, after disconnecting a bag that has been filled, cryogenically preserving the cells in the bag, for example, in liquid nitrogen according to known methods.

In another embodiment the invention method comprises the steps of: (a) providing the cells suspended in a liquid; (b) providing a plurality of sterile flexible bags connected to a main line of sterile flexible tubing by branch lines; (c) evacuating air from the flexible bags by applying a vacuum to the main line; (d) preventing fluid flow through all branch lines except that of one bag to be filled; (e) dispensing a desired volume of the cell suspension into the main line; and (f) forcing purging gas into the main line to drive any residual cell suspension liquid from the main line through the branch line into the bag to be filled.

More in particular, the cells used in the present invention are typically mammalian cells, usually human cells to be used as a therapeutic product, such as human stem cells. Typically, the cells are suspended in a liquid medium formulated for storage of the cells, for instance, for cryopreservation in liquid nitrogen using DMSO or other known cryopreservation agents.

In another aspect, the invention also provides pre-sterilized flexible bag sets or manifolds, for filling sterile, flexible bags according to the invention method. These bag sets comprise a plurality of sterile flexible bags fluidly connected to a main line by a plurality of branch lines. This main line is a sterile length of flexible tubing having an open end available for fluid flow in or out of the main line; the other end of the main line may be sealed or connected to a branch line. Each branch line is a sterile length of flexible tubing having one end fluidly connected to one of the bags in the set and the other end fluidly connected to the main line. In some embodiments of the invention, multiple main lines may be independently connected to one cell source container, to fill multiple bag sets in parallel, thereby further enhancing the bag filling rate for a single large batch of cells.

Bags sets suitable for use in the present invention are disclosed, for instance, in U.S. Pat. Nos. 6,712,963 and 7,052,603, and U.S. Pat. App. Pub. No. 2006/0118472, cited above, and be assembled using commercially available flexible bag manifolds such as PEDI-PAK® Quad 75 mL Transfer Packs, from Genesis BPS, 65 Commerce Way, Hackensack, N.J. 07601. Bag sets of the invention also may be assembled by connecting any other flexible bags suitable for cryogenic storage of mammalian cells with main and branch lines comprising suitable lengths of appropriate flexible tubing sterilely welded into the desired manifold arrangement.

According to the invention, contrary to conventional methods, air is evacuated from the flexible bags prior to filling with the mammalian cell suspension to be cryopreserved. In particular, the method involves evacuating air from at least one of the flexible bags in a manifold by applying a vacuum to the open end of the main line. Typically, vacuum is applied using a laboratory grade vacuum pump. The level and duration of vacuum application may be varied as needed to provide sufficient removal of air in a convenient time period, according to the invention, as further illustrated in the Examples below. Vacuum may be applied to a single bag prior to filling that bag or, advantageously, to all bags at one time.

After evacuating air from the bags via the main and branch lines, the invention method involves preventing fluid flow through all branch lines except that of one bag to be filled. Generally, the fluid flow in a main or branch line is controlled by a valve that selectively allows or stops fluid flow in the flexible tubing. Pinch valves are advantageously used for this purpose because such valves do not breach the sterile integrity of the flexible tubing. Typically, fluid flow is prevented in a branch line by applying a pinch clamp valve to the tubing near its junction with the main line, thereby advantageously providing minimal entry of liquid into the closed branch line.

After preventing flow in all branch lines except that of the one bag to be filled according to the invention method, a desired volume cell suspension liquid is dispensed into the open end of the main line. This volume may be dispensed, for instance, manually with a syringe or by any motor-driven pump. Advantageously, the desired volume of cell suspension is dispensed with a peristaltic pump so that no pump mechanism directly contacts the suspension, again minimizing chances of microbial contamination by maintaining the flow through closed sterile tubing. The flow of liquid into the main line may be stopped after the desired volume is dispensed by stopping the pump and/or closing the open end of the main line with a valve, for instance, a pinch valve. The pump motor or mainline valve may be stopped, for instance, by a remote controller that detects the weight of liquid dispensed into the bag set or remaining in the cell source container. Alternatively, the controller could monitor the pumping time to stop dispensing after a desired volume is pumped.

To reduce waste and provide better consistency of filling volume, the invention also provides for purging of residual cell suspension from the main and branch lines after the desired volume is dispensed into the bag set via the open end of the main line. Thus; in this aspect of the method, sufficient sterile purging gas is introduced under pressure into the open end of the main line to drive into the one bag to be filled any dispensed liquid remaining in the main line or the open branch line of that one bag. Conveniently, the sterile purging gas is filtered air, but other inert gases, such as nitrogen, also may be used, as known in the art for cryogenic preservation of live mammalian cells. The flow of purging gas may be stopped by a valve when all residual liquid is forced into the bag to be filled. The flow of purging gas may be controlled by a valve between the purging gas source and the mainline, but typically is controlled by pinch valve on the main or branch line of the bag to be filled. Advantageously, the valve stopping the flow of purging gas is remotely controlled by a controller that detects removal of all residual liquid from the main and branch lines of the bag to be filled, e.g., by weight of the filled bag or passing of the trailing meniscus of the residual liquid past a detector located close to the connection of the branch line to the bag to be filled, thereby simultaneously minimizing product loss in the tubing and undesired entry of purging gas into the filled bag.

After filling, the invention method provides for sterilely sealing and disconnecting the branch line of a bag that has been filled, typically by sterilely welding closed and cutting the branch line tubing. Bags may be serially removed after each is filled, or removed together after all bags are filled.

Although pinch clamp valves are advantageously used in the invention method and apparatus to minimize breaches in the sterilized tubing, the invention also contemplates use of in-line valves in some circumstances. For instance, it may be desirable for simplifying automation to use a single three-way in-line valve for selectively connecting the cell suspension source, vacuum source or purging gas source to the mainline of a bag set, particularly were a disposable plastic in-line valve is available.

In another aspect the invention method of storing live mammalian cells comprising dispensing the cells into sterile, flexible bags according to the above method of the invention, and then cryogenically preserving the cells in filled bags, sealed bags.

Yet another aspect of the invention provides an apparatus for aseptically dispensing live mammalian cells into sterile, flexible bags in a non-sterile atmosphere, according to the invention method. In some embodiments, this apparatus comprises: a plurality of sterile flexible bags, a cell source container configured to contain the live mammalian cells suspended in a liquid, a vacuum source; and a sterile purging gas source. In these embodiments, the plurality of flexible bags, cell source container, vacuum source and gas source are fluidly connected together to form a sterile system that is closed to the external environment such that the system selectively allows fluid flow between the sterile bags and either the cell source container or the vacuum source or the gas source. In some embodiments, the plurality of flexible bags, cell source container, vacuum source and sterile purging gas source are fluidly connected together at least in part by flexible tubing; and at least one pinch valve that externally engages the flexible tubing is provided to selectively allow fluid flow between the sterile bags and the cell source container or the vacuum source or the gas source. This apparatus may also further comprise a peristaltic pump unit configured to pump liquid from the source container through the flexible tubing into the flexible bags, and advantageously either the pump unit or one or more pinch valves, or both, are remotely operable by a controller.

In a particular embodiment of this apparatus, the flexible tubing comprises a main line and at least one branch line, where the main line is a length of flexible tubing having one end connected to the cell source container and the purging gas source so as to selectively allow fluid flow into the main line from the container or the gas source. In this embodiment, each of the flexible bags is fluidly connected to the main line by a branch line, with each branch line being a length of flexible tubing having one end fluidly connected to one of the bags and the other end fluidly connected to the main line so as to allow liquid or gas to flow from the main line into the bag. Further, the vacuum source is fluidly connected to the main line so as to allow the vacuum source to withdraw gas from each of the bags through the mainline and branch lines.

All interior surfaces of this apparatus that come in contact with the liquid from the cell source container are pre-sterilized, and the apparatus is designed to exclude non-sterile air from the surrounding atmosphere. For instance, a sterilizing filter may be used in any opening to the outside of the apparatus that may be needed to allow entry from, exit to, or exchange of gasses in the non-sterile environment of the apparatus. The invention apparatus therefore provides a sterilized, disposable system that is completely closed to external microbial contamination, for aseptically filling flexible bags with live mammalian cells, for instance, for cryogenic preservations.

Advantageously, the cell source container used in the invention is disposable, typically a pre-sterilized flexible bag which can be aseptically filled by sterilely welding flexible tubing to the container. In the apparatus, the container is connected to a main line which is a length of flexible tubing with one end fluidly connected to the container, either directly or by connecting to another length of tubing already connected to the container. In any even the main line tubing is connected to the container so as to allow liquid in the container to flow into the main line.

The invention apparatus also includes bag set or manifold, which is a plurality of flexible bags to be filled with cell suspension, each of which is connected to the main line by a branch line. Each branch line is a length of flexible tubing having one end fluidly connected to one flexible bag and the other end fluidly connected to the main line, so as to allow liquid to flow from the container into the bag. The number of bags in bag sets of the invention may be from 2 to over 1000, such as 3, 4, 5, 8, 10, 20, 30, 40, 50, 60, 70, 80, 90, 100, 200, 400 or 800, preferably from about 10 to about 100. Suitable bag sets and methods of assembling such from commercially available materials are described hereinabove.

The invention apparatus further comprises a vacuum source selectively connected to the bag set so as to allow vacuum to be applied to each bag in the set, either all at the same time or individually. The vacuum source is used to evacuate the bag set prior to filling, for instance, to avoid problems with cryopreservation of cells in filled flexible bags containing too much air which interferes with achieving a consistent cooling rate throughout the cell suspension.

The apparatus further provides a sterile purging gas source that is fluidly connected to the bag set so as to allow such gas to flow from the gas source to each of the bags, driving any dispensed liquid remaining in the main or branch line tubing into the bag to be filled, as described for the filling method of the invention, above. Advantageously, the gas source is selectively connected to the bag set, for instance, to a main line, near the connection of the cell source container, so that the purging gas enters close to where the liquid enters the system and therefore clears most of the flexible tubing path between the cell source and the bag to be filled.

In some embodiments, the apparatus of the invention further comprises a valve that selectively allows or stops fluid flow through one of the lengths of flexible tubing. Preferably, such valve is a pinch valve that externally engages flexible tubing of the bag set to selectively allow or stop fluid flow in the tubing without breaching the sterilized tubing. Further, the apparatus advantageously comprises a peristaltic pump unit located so as to allow pumping of the liquid from the source container through the main line and branch lines into the bags.

The entire apparatus and method of the invention allow filling of sterile bags in a completely closed sterile system, and are also readily adapted to partial or total automation using remote control of pumps, valves and sterile tubing welding, according to methods well known in the art of process control and automation. The invention thereby greatly improves production rate and quality compared to previous, largely manual processes for aseptically dispensing live mammalian cells into sterile, flexible bags, for instance, for cryopreservation in liquid nitrogen.

### DESCRIPTION OF THE FIGURES

FIG. 1 shows a schematic of an apparatus used in methods of the invention for filling a single final product bag;

FIG. 2 shows a schematic of an automated closed bag-filling apparatus of the invention, for filing five final product bags in a bag set or manifold-unit (PD=Product Dose);

FIG. 3 shows that residual air volume in a filled bag depends on the level of applied vacuum prior to filling according to the invention;

FIG. 4 shows the effect of vacuum duration (commercial vacuum pump attached to a laboratory house line) on residual air in a final product bag filled according to the present invention;

FIG. 5 shows the effect on packaged fluid volume of purging residual fluid from fill line into the final product bag using air pressure;

FIG. 6 shows that peristaltic pump speed and tubing size has no substantial effect on the viability of cells during product bag filling according to the present invention, over the tested range of pump velocity (100-400 rpm) and tubing sizes (0.8 to 3.2 mm ID);

FIG. 7A shows the effects of pump speed and tubing size (inside diameter) on viability of cells at 3.0 million cells/mL;

FIG. 7B shows the effects pump speed and tubing size (inside diameter) on viability of cells at (B) 10.0 million cells/mL;

FIG. 8 shows that maintaining a uniform suspension of source cells during the filling process of the invention may be readily accomplished by mechanical agitation of the source suspension; and

FIG. 9 shows results of a large scale test of the filling process of the invention, with 20 product bags being filled consecutively using both the evacuation and purging steps of the process.

### DETAILED DESCRIPTION OF THE INVENTION

The present invention provides improved methods, and associated apparatus and systems for automated performance of those methods, for efficiently, reliably and safely filling final product bags with cell therapy products containing live mammalian cells. In current processes for manufacturing cell therapy products, filling the final product container, typically a flexible cryogenic storage bag, is the current production bottleneck—even at small lot sizes of less than 100 final product bags. The main time constraint at this stage of down-stream processing is the limited period after addition of the cryoprotectant, DMSO, that mammalian cells generally will retain acceptable viability, which is typically only about 60 to 90 minutes.

The traditional bag-filling process is a complex multi-step, manual process, requiring use of a syringe for filling and evacuation of air after filling. See, e.g., U.S. Pat. No. 6,136,525 on a “Method of Cryopreserving Hepatocytes,” cited above. To reduce potential for microbial contamination while connecting and disconnecting the syringe from the cell source and the final product bag, the source, product bag and syringe may be provided with integrally connected pieces of tubing that can be spliced and separated via sterile tubing welding. See, for instance, sterile tubing welding processes and devices disclosed in U.S. Pat. App. Pub. No. 20070142960, on a “Sterile Tubing Welder System,” published on Jun. 21, 2007, by Bollinger et al., and other patent documents cited therein. Sterile welding creates sterile tubing connections while maintaining a functionally closed system, thereby minimizing contamination even in non-sterile environments.

In a traditional bag-filling process previously used by the present inventors, for instance, a syringe is sterile welded to a source bag containing a liter or more of a final cell therapy product comprising therapeutic cells suspended in a cryoprotective medium containing DMSO. The cells in the source bag are suspended at a pre-set density, and the syringe is filled with a pre-set volume, both of which are product-specific. Each syringe is then separated from the source by severing its attached tubing with a tubing sealer, and that tubing is then sterile welded to the tubing attached to a final product container, typically a 50 mL Cryocyte® flexible freezing bag (Baxter Healthcare Corporation, Irvine, Calif.). The final product, typically 5-20 mL of cell suspension, is then injected into the bag, the bag is inverted and the syringe is used to remove any residual air pocket or bubbles from above the cell suspension, carefully avoiding inadvertent removal of any final product suspension. Finally, the syringe tubing is then disconnected with a tubing sealer, and the bag is ready for freezing. Throughput of such a manual process, using six trained technicians, is about 60 bags per hour, allowing processing of a batch size of about 1.2 liters for a 20 mL/bag dosage, and only about 0.3 liters for a 5 mL dosage, within the preferred 60 minute window for freezing cells after adding DMSO.

The present inventors have analyzed the above traditional process for filling bags with therapeutic cells in cryoprotectant and discovered several improvements that facilitate efficient automation of a process completely closed to external contamination, leading to greater throughput and reproducibility of final product dosage. In initial attempts to enable automation of the instant process, however, the present inventors discovered that using a peristaltic pump to introduce the cell suspension into conventional cryogenic freezing bags (e.g., a 50 mL Cryocyte® bag) left unacceptable amounts of air in the bags, coming from both bag manufacturing and the tubing used for filling. In the prior manual process described above, this residual air was removed using the filling syringe. As noted elsewhere herein, the existence of air bubbles or pockets in a cryogenic freezing bag is highly undesirable. For instance, the Cryocyte® bag filling instructions (found at www.cryocyte.com) state multiple times that care must be taken to remove all air from the filled bag prior to cryopreservation. Air bubbles or pockets can cause non-homogeneous cell volume distribution leading to lack of controlled freezing, and it can also lead to bag fracture from mechanical agitation at very low temperatures obtained in liquid nitrogen storage systems.

Another problem the inventors found when filling flexible bags with final cell product by pumping with a peristaltic pump was that substantial loss of product could occur due to fluid within the tubing connecting the product source to the fill bag. Pumping product from a large product source generally will lead to more product being retained in the tubing compared to the prior manual method, as practical considerations are likely to dictate longer tubing to connect a large source to a small filled bag than to connect the previously used syringe to such a bag. Thus, some loss of final product in the filling tubing is inevitable in a peristaltic pump-based filling system, but minimizing this loss is necessary for efficient production.

In addition, the present inventors discovered that a major rate-limiting activity in the above manual bag-filling process is the necessary performance of multiple sterile welds to fill a syringe and empty it into a single bag, as current sterile welding processes typically require several minutes to complete each weld.

Accordingly, to address the challenge of efficient automated filling of cryogenic freezer bags with cells, including reducing residual air in the bags, while minimizing the number of sterile welds and product loss, the present inventors have developed a novel process that removes at least about 95% of the air that typically remains in a freezer bag after filling with a syringe or pump, without opening the system or incorporating more sterile welding. This process simply requires one sterile weld to the final product bag, and after filling and sealing, the bag can immediately be packaged for cryopreservation or cold storage.

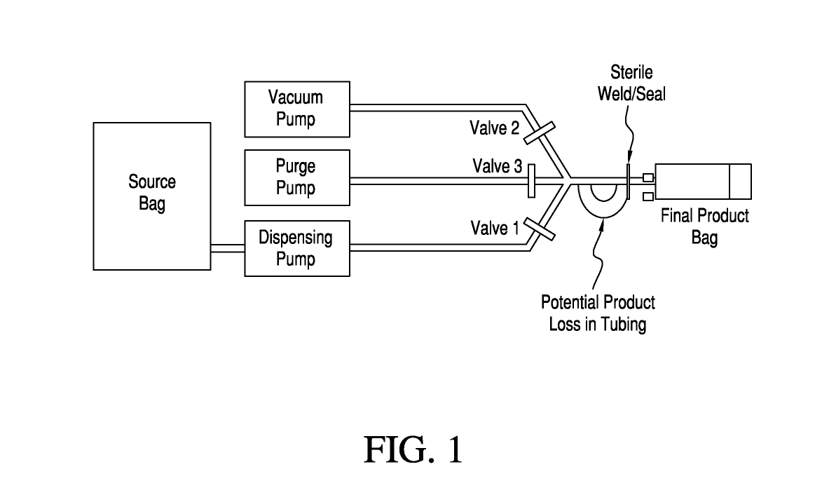

In one embodiment, therefore, the novel process involves connecting a final product bag (e.g., a Cryocyte® cryogenic freezing bag) via a sterile plastic tubing network to at least (a) a cell source container, preferably a flexible bag containing cells suspended at the final product density in cryoprotectant medium which is delivered from the source to the product bag via a dispensing pump; and (b) a vacuum source. Optionally, the final product bag is also connected via the sterile tubing network to (c) a purging gas source, such as a source of sterile pressurized air. A schematic of a simple apparatus for performing the novel process of the invention for filling a single final product bag is shown in FIG. 1. The sterile tubing network of this apparatus of the invention including the purging gas source has at least three valves, such as pinch valves, which can open and close the vacuum, purge, and cell source lines to the final product bag to sequentially: (1) remove air from the final product bag and tubing network; (2) fill the final product bag with a desired amount of final product; and (3) purge the tubing network of retained product fluid, forcing that fluid into the final product bag, thereby minimizing product loss in the tubing.

Using the apparatus in FIG. 1, the process of the invention may be conducted according to the following guidance: (1) Assemble source bag, dispensing pump, vacuum pump and purge pump; (2) Sterile weld the Final Product Bag; (3) Close valve 1 and open valve 2 to allow evacuation of air from the final product bag and tubing for a predetermined (optimized) time; (4) Close valve 2 and open valve 1 to dispense product specific-volume; (5) Close valve 1 and open valve 3 to allow sufficient purge fluid (e.g., sterile air) to force residual product from the tubing into the final product bag; (6) Close valve 3 before purge fluid enters the final product bag; and (7) Sterilely seal and cleave the tubing to the filled bag which may then be packaged for cryopreservation or cold storage.

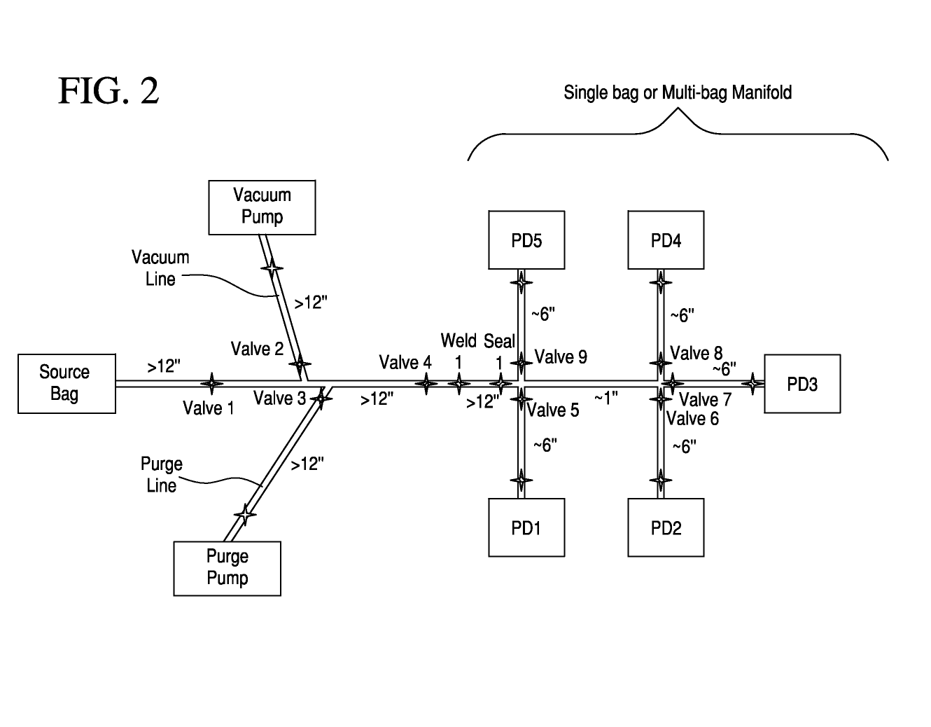

To increase the throughput of the apparatus and bag-filling methods of the invention, for more efficiently filling multiple final product bags, the present inventors also designed flexible bag sets (also referred to in some embodiments herein as “multi-bag manifold units”) in which each bag is connected by tubing to a common main tubing section such that fluid introduced into one end of the common main tubing can flow into each of the connected bags. A schematic of a simple apparatus for conducting methods of the invention to fill five final product bags in a multi-bag manifold unit is shown in FIG. 2.

In a multi-bag manifold unit as shown in FIG. 2 for the filling of multiple bags, the process may be conducted as follows: (1) assemble source bag, dispensing pump, vacuum pump and purge pump; (2) Sterile weld Final Product Bag as shown in FIG. 2; (3) Close valve 1 and open valve 2 to allow evacuation of air from the final product bag and tubing for a predetermined (optimized) time; (4) Close valve 2 and open valve 1 to dispense product specific-volume; (5) Close valve 1 and open valve 3 to allow sufficient purge fluid (e.g., sterile air) to force residual product from the tubing into the final product bag; (6) Close valve 3 before purge fluid enters the final product bag; and (7) Sterilely seal and cleave the tubing to each of filled bag which may then be packaged for cryopreservation or cold storage.

Each of the above steps in repeated in sequence for each of the final products bags. In another embodiment the filling may be performed in parallel such that the product bags are all filled in parallel as discussed above and thereafter sealed and cleaved, in sequence or in parallel.

Manifolds of disposable tubing and flexible bags, suitable for practicing the present invention methods using a peristaltic type of pump, together with one or more remotely operated pinch valve(s) that are operated by a controller to provide automated delivery of fluid into such bags of a biotechnology fluid in an aseptic environment, are known. See, for instance, U.S. Pat. Nos. 6,712,963 and 7,052,603, and U.S. Pat. App. Pub. No. 2006/0118472, cited above, disclosing use of flexible bag manifold units, assembled via aseptic connectors, in automated methods for dispensing biotechnology fluids such as chromatography eluates. Typical, flexible bags known as PEDI-PAK® Quad 75 mL Transfer Packs, are commercially available from Genesis BPS, 65 Commerce Way, Hackensack, N.J. 07601.

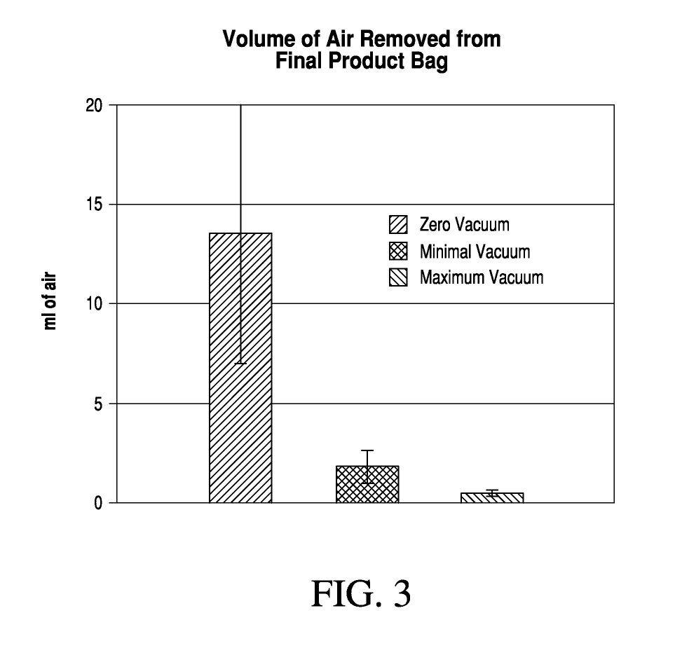

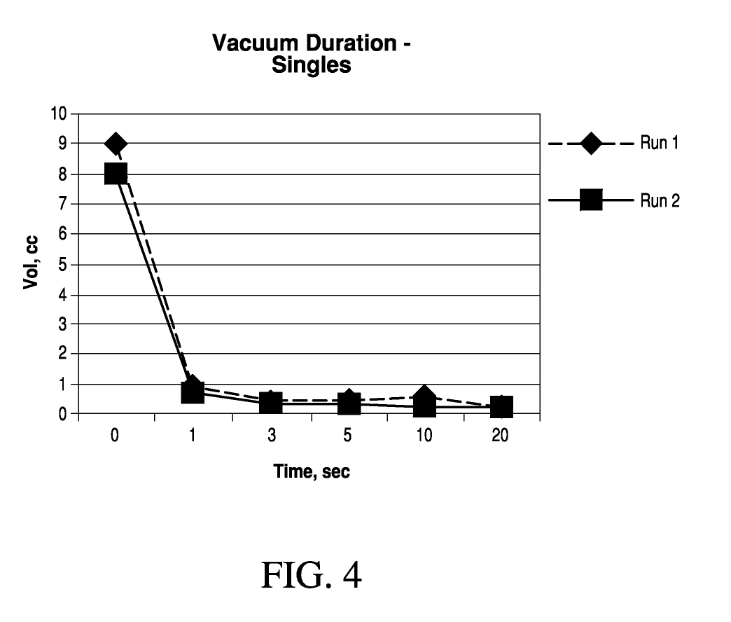

To demonstrate proof of concept for adequate air removal from cryogenic bags before filling with cells, an apparatus according to FIG. 1, without the optional purge pump, was initially tested. As described in Example 1, below, the inventors found that the amount of air left in the bag is dependent on the level of vacuum applied prior to the filling step. FIGS. 3 and 4 show data from experiments where the amount of residual air in an evacuated bag was quantified after filling by withdrawing residual air into a syringe. The results demonstrate that various vacuum sources and application periods can sufficiently evacuate air in the bags prior to filling, with application of an ordinary laboratory house vacuum line for a few seconds being sufficient to remove at least about 95% of residual air. Although not generally necessary, additional air removal may be achieved before bag filling, by applying a stronger vacuum source or by applying the vacuum for longer times (both removing air more effectively), or after bag filling, for instance, by further application of vacuum, either with or without manipulating the bag, manually or by mechanical means, to drive trapped air bubbles to the top of the bag.

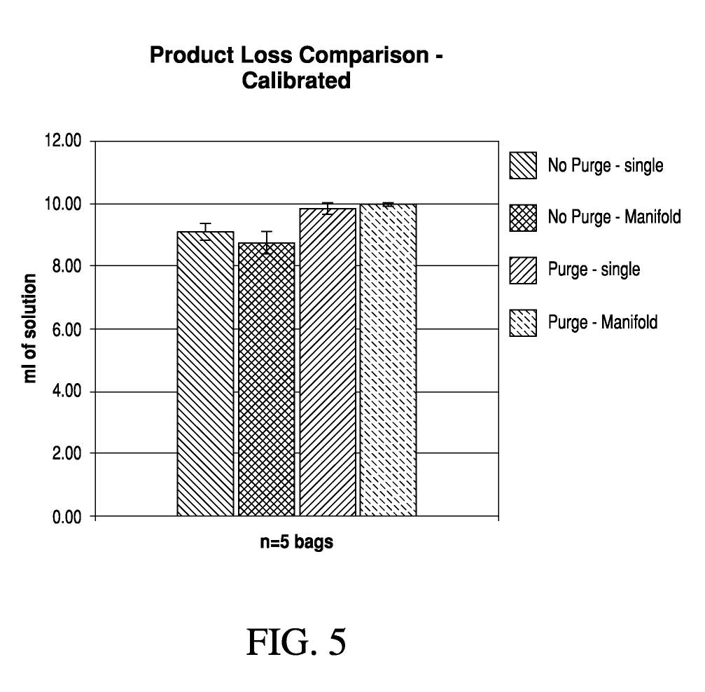

Regarding loss of product retained in the filling tubing, in single bag tests with an apparatus of FIG. 1, the inventors observed that over 1 mL of product was lost during filling, which represents from 5% (for a 20 mL dosage) to up to 20% (for a 5 mL dosage) of final product loss, which is unacceptable for these inherently expensive products. See Example 2, below. While such product loss can be reduced by minimizing tubing lengths as much as possible using readily available components of the apparatus, as illustrated in FIG. 5, this product loss also can be dramatically reduced via the addition of a purge line. The additional step of purging the tubing with air was found not only to reduce the loss of product, but also to decrease the variability (standard deviation) of the product dosage in the final product bag, to as low as +/−0.5% (see Example 2), thereby creating a more robust process with tighter dosage specifications.

Importantly, this purge step is simply automated and controllable through disposable tubing and bag sets coupled with control logic and valves, plus sensors that close the purge source valve so as to prevent excess purging fluid from entering the filled bag, for instance, by detecting when the bag contains a complete dosage (by weight) or when the interface between the purging fluid and the product fluid passes a point near the filled bag, using sensors that are known and readily available for automation of fluid dispensing processes. Thus, the process of the invention can be fully or partially automated using an apparatus according to FIG. 2, for instance, with manual or computer control of the vacuum, dispensing pump, purge pump, and valves, as well as other devices and disposables that are designed around the invention to provide complete bag filling systems. Computer control of all valves and pumps would be operated via simple control logic software, and would be particularly useful in filling tens of bags on each of several individual manifold units. This would greatly increase the bag filling throughput of therapeutic cell manufacturing and further relieve a critical manufacturing bottleneck.

The bag filling process and apparatus of the invention provide substantially greater throughput with less labor in filling cryogenic bags with cell therapy products, compared to the previously used manual process. As described above, throughput of that manual process, using six trained technicians, is about 60 filled bags per hour, allowing processing of a batch size of about 1.2 liters for a 20 mL/bag dosage, and only about 0.3 liters for a 5 mL dosage, within the preferred 60 minute window for freezing cells after adding DMSO. As set for the in Example 4, below, the present inventors have estimated that, with a 18 mL dosage, for instance, in one hour a single technician could fill only 65 single bags compared to 139 bags using a 3-bag manifold. Although this estimate does not include all effort required under actual production conditions, it is clear that the present invention can dramatically increase throughput per hour of technician effort, from about 10 filled bags to over 100 bags per hour, thereby allowing for processing of a 1.2 liter batch by a single technician or processing of much larger batches by multiple technicians, each using a separate apparatus (particularly, pump and tubing welder).

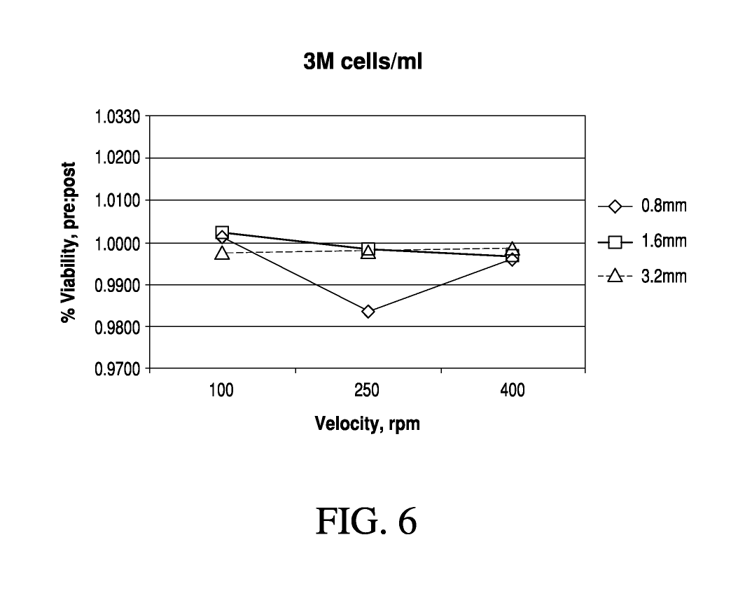

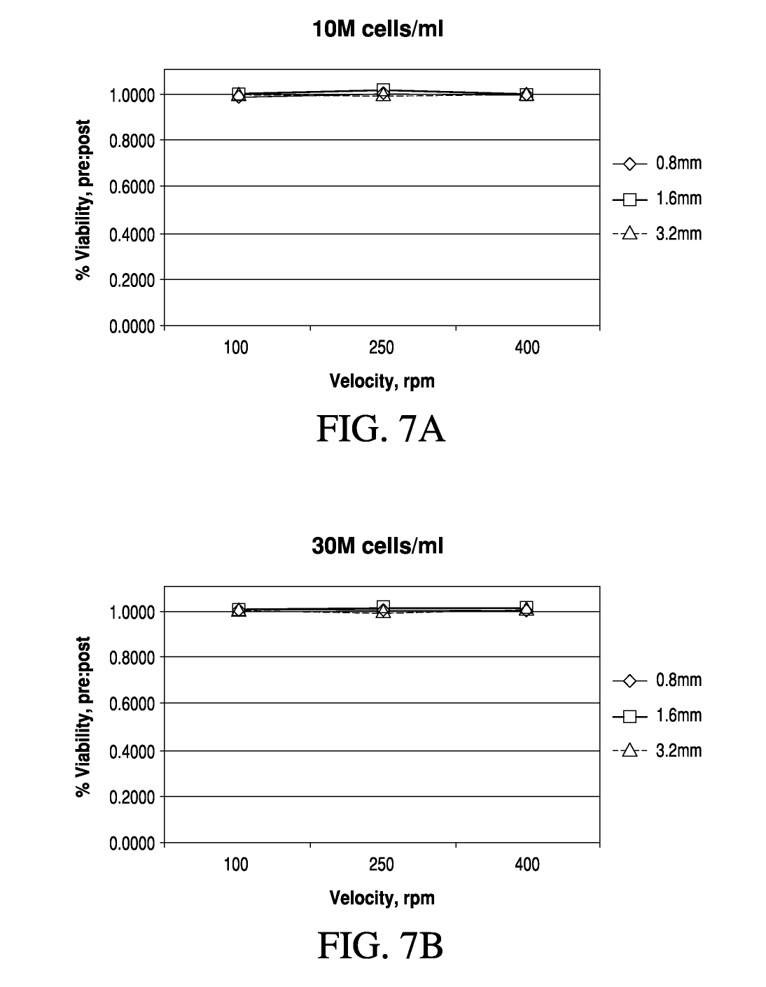

The inventors have also investigated other operational parameters of the bag-filling process of the invention. For instance, Example 4 describes test results, presented in FIG. 6, showing that the peristaltic pump speed and tubing size has no substantial effect on the viability of cells during product bag filling according to the present invention, over the tested range of pump velocity (100-400 rpm) and tubing sizes (0.8 to 3.2 mm ID). Example 5 describes more extensive testing of effects on cell viability of tubing size, pump velocity and cell concentration over a range representative of typical product concentrations (1 to 30 million cells/mL). As shown in FIGS. 7A and 7B, none of the tested parameters significantly affected cell viability over the tested ranges.

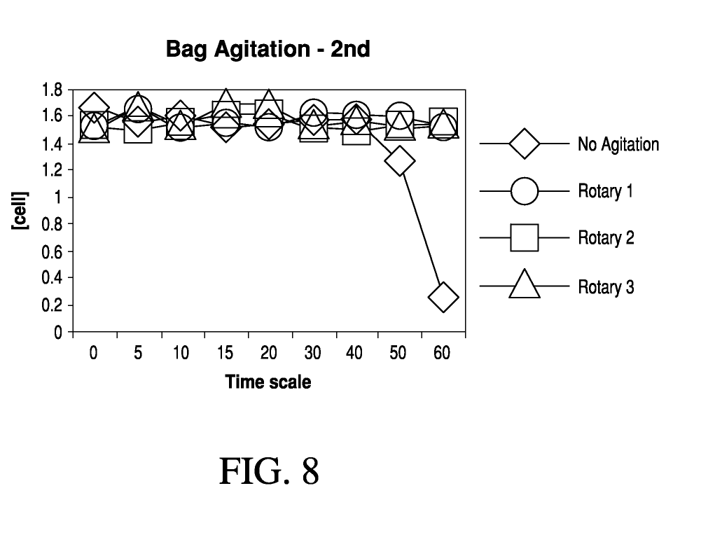

In addition, testing in Example 5, with results shown in FIG. 8, demonstrates that maintaining a uniform suspension of source cells during the filling process of the invention may be readily accomplished by mechanical agitation of the source suspension. The inventors have also examined the effect of the cryopreservative, DMSO, on cell viability during the bag-filling process of the invention. Tests described in Example 6 showed no significant affect of DMSO upon viability of human dermal fibroblast (HDF) cells that are dispensed by pumping through tubing in a timely manner, according to the bag-filling process of the invention.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Removal of Residual Air from Flexible Bags

It is known that residual air in a cryogenic freezing bag has a negative impact on viability of frozen cells and the physical integrity of the bags after freezing in liquid nitrogen. The following tests were performed to determine the effectiveness of the present invention process for removal of residual air from cryogenic freezing bags.

### Materials and Equipment

Pedi-Pak flexible 75 mL sample bags (p/n 402-04, Genesis BPS, Commerce Way, Hackensack, N.J. 07601)
60 mL syringe (p/n 06009)
18 ga needle (p/n 08344)
Hemostats
Water
Top loading scale
Low pressure vacuum (commercial vacuum pump)
High pressure vacuum (simulated with syringe)
Flexicon Peristaltic Pump—DF6

The testing used 75 mL flexible Pedi-Pak® Bags to simulate final product cryogenic freezing bags. Control bags were used directly as received from the manufacturer. Test bags were exhumed of residual air by one of two means. The first consisted of attaching a 60 mL syringe to the luer-lock port of the bag and manually pulling on the plunger until no more air could be removed. The second consisted of attaching the luer-lock port to the laboratory house vacuum line (commercial vacuum pump) and allowing the bags to be evacuated for not longer than one minute. After evacuation, bags were sealed and then sampled for remaining air volume. The volume of air remaining was measured by introducing 15 mL of water into the bag, which was sufficient to allow the remaining air to rise into the sampling port such that a syringe with 18 ga needle could be inserted into the port to withdraw the air. The volume of air in the bag was estimated from the volume that could be removed in the syringe without removing water.

As illustrated in FIG. 3, the testing results (n=5 bags for each condition) showed that residual air of control bags (no air removed; zero vacuum) averaged 13.5 mL±6.5 mL, whereas the residual air after low vacuum (syringe; minimal vacuum) averaged 1.8 mL±0.8 mL, and after high vacuum (house line; maximum vacuum), 0.425 mL±0.1 mL. The residual air in evacuated bags was deemed to be within acceptable levels for cryopreservation, with application of the higher vacuum being preferred as it removed about 97% of the residual air remaining in control bags that were not evacuated.

Further testing of the effects of evacuation by applying vacuum via a commercial vacuum pump for various time periods, from 0 to 20 seconds, showed that maximum removal of residual air (about 97%) was achieved in about 3 seconds. See FIG. 4.

The above tests demonstrate that performing air removal prior to filling bags with product (opposite to current practice), using the filling process of the invention with a closed sterile system, provides an adequate method of air removal to prepare the bag for closed system filling. Alternatively, according to the present invention, air could be removed from bags by the manufacturer and supplied without air for filling without the integrated vacuum step of the present process.

### Example 2

### Purging of Product Fluid from Tubing Used to Fill Bags

Product loss during production occurs in many stages including fluid remaining in tubing during product bag filling. Relatively small amounts of solution accumulate into substantial sums when production scales are increased to commercial levels. Furthermore, every mL of final product is extremely valuable for inherently expensive cell therapies, and even a 5% product loss can cost hundreds of thousands of US dollars in final product lost during large lot processing (e.g., 500-1000 product doses). The inventors have therefore developed a process for purging product fluid from the tubing lines into the filled bags prior to sealing.

### Materials and Equipment

Flexicon Peristaltic Pump—DF6

Flexible tubing, 3.2 mm inner diameter (ID)

Top loading scale

Pedi-Pak® Bags (see Example 1)

60 mL syringe with 18 ga needle

Water

Testing of the purging process consisted of using standard conditions on the Flexicon Pump to dispense 10 mL of water into 5 single bags and into bags in a 5 bag manifold. The control conditions consisted of no purge or manipulation of the fluid in the tubing. The test conditions consisted of using a ‘Y’ connector to add a 60 mL syringe to the fill line with a clamp to close the line to the syringe. After each bag was filled, the clamp to the syringe was released and sufficient air was expelled to push fluid remaining in the fill line to within about 3 mm of the product bag. The tubing to the bag was then clamped and the next bag was filled with the same procedure. A syringe with needle was then used to remove the fluid from each bag. The fluid from each bag was weighed, and the weights were recorded and analyzed to determine an average delivered dosage per bag.

As shown in FIG. 5, the average weight of single bags with no purge was 9.09 g±0.26 g and the average weight of a bag from the five bag manifold with no purge was 8.76 g±0.33 g. In contrast, the average weight of a single bag with the gas purge process was 9.85 g±0.17 g while the average weight of one bag from the 5 bag manifold was 9.97 g±0.05 g.

The results of this test therefore showed that purging the tubing lines can save over 1 mL (or 10%) of product per bag. It also showed the degree of inaccuracy dropped considerably from as much as a ±0.33 g (3.3%) to as low as ±0.05 g (0.5%). Such savings of product retained in the fill lines will substantially increase efficiency and reduce costs and time money for large scale production.

### Example 3

### Analysis of Process Times and Effects on Throughput

The process of manually filling individual product bags consumes substantial amount of effort by highly trained technicians. The inventors have separately analyzed the effort required for individual acts of sterile welding a bag to the pump assembly, filling the bag, sealing the bag, and removing it from the fill line. Based on these analyses, a strategy has been developed for efficient bag-filling, using multi-bag manifolds to eliminate many of the sterile welds required in the manual process.

### Materials and Equipment

Flexicon Pump—DP6

Terumo Sterile Tubing Welder

Hand Tubing Sealer (heated)

Timer

Pedi-Pak® Bags

Water

The estimated total time to fill each bag in a single bag system versus a multi-bag manifold was calculated by determining the average elapsed time for individual steps of the process and then adding these to estimate the total process time. The fill process is broken into several steps listed below:

Welding

Alignment of tubing in welder

Welding of tubing

Inspection of weld

Heat sealing of tubing

Detachment of tubing from bag

Filling—the actual time of dispensing fluid

The filling time does not change per bag whether single bags or multi-bag manifolds are used. For per bag welding time of multi-bag manifolds, the alignment, welding, and inspection of the single weld required between the cell source line and the common main input line of the manifold is averaged over the number of bags on the manifold. Sealing and detaching times per bag are the same for each bag, whether single or in a manifold. These average step times are then added to calculate the average, expected time to fill one bag separately or in a manifold.

The collected data produced the following averages:

Align: 8.0 sec Seal: 19.0 sec

Weld: 19.6 sec Detach: 2.0 sec

Bag Seal Test: 3.0 sec

These numbers indicate that the complete tubing connect and disconnect process for a 3 bag manifold would take 31.2 sec per bag, for a 5-bag manifold, 27.12 sec, and for a 7 bag manifold, 25.37 sec. When combined with the fill time (˜2 sec for 5 mL and ˜4 sec for 18 mL), it can be estimated that, with a 5 mL dosage, in one hour a single technician could fill only 67 single bags compared to 112 bags using a 3-bag manifold. Additional estimates for other fill volumes and manifold sizes are show in the following Table 1.

TABLE 1
Estimated bags filled in one hour by one technician

Volume Dispensed:

| | 5 mL | 18 mL |
|---|---|---|
| Single Bag | 67 | 65 |
| 3 Bag Manifold | 112 | 110 |
| 7 Bag Manifold | 140 | 139 |

Note that the above estimated process times do not include times for preparation of product cell suspension or set up of the apparatus. Nevertheless, use of multi-bag manifolds according to the present invention can greatly reduce the time and effort required to fill final product cryopreservation bags. Alternatively, multiple fill lines from a single product source (manifolded or not) could be utilized to increase the throughput of bag filling to several hundreds to thousands per hour.

### Example 4

### Effects of Pumping Speed on Cell Viability

As current bag filling operations are performed manually with syringes, the inventors have investigated the issue of whether dispensing cells into product bags at high speeds, through flexible tubing with a peristaltic pump, would decrease cell quality. A full factorial experiment testing the three main variables governing cell quality post-dispense (pump speed, tubing size, and cell concentration). Over the tested range of pump speeds and tubing sizes, the pumping velocity was shown to have little effect on the viability of cells (regardless of cell concentration) during product bag filling according to the present invention.

### Materials and Equipment

Human Dermal Fibroblasts (HDF)

Medium—Plasmalyte A (P/N 07204)+5% Human Serum Antibody (P/N 07489)

Flexicon Pump—model DF6

Tubing sets—0.8, 1.6 and 3.2 mm ID

Centrifugal tubes—various

Nucleocounter®

Nucleocassettes®

Buffer (PBS—P/N17-516F)

The testing used HDF cells suspended in a maintenance medium (without DMSO) at a density of 10.0 million cells/mL. The pump parameters were as follows: volume=2 mL, acceleration=100 rpm, reverse=1.0. Tubing size in separate tests was 0.8, 1.6 or 3.2 mm inside diameter. These tubing sizes were tested as they are appropriate for the range of filling volumes ranging from hundreds of microliters (0.8 mm tubing) to >50 mL fills (3.2 mm tubing). The pump velocity was set in each separate test to 100 rpm, 250 rpm, or 400 rpm, resulting in the following pumped volume rates for the 0.8, 1.6 and 3.2 mm ID tubing sizes, respectively, of 25, 50 and 240 mL/min at 100 rpm, and 60, 140 and 720 mL/min at 400 rpm. Accordingly, the tested range of pumped flow volumes in the present example was about 25 mL/min to abut 720 mL/min.

The source container was a 50 mL centrifuge tube that was manually agitated during the process to maintain a uniform density of the cells in suspension. The collection container was a 50 mL centrifuge tube. The tubing set was primed prior to start each run. Prior to collection of each sample, 5 doses were dispensed and discarded, to clear the tubing of cells that may have adhered or clumped during pauses in dispensing. Samples were collected in separate 15 mL centrifuge tubes. Cell quality was evaluated by assaying for cell viability. 200 μl of each was transferred to a micro-tube containing 400 ul of PBS to provide the cell concentration needed for viability testing. Each sample in PBS analyzed on a NucleoCounter® cell counter % viability. In this instrument cell viability is determined by propidium iodide dye exclusion. The cells were reused for each run.

As shown in FIGS. 6-8, the resulting data showed less than 0.8% loss of average % viability across the three speed variables over a range of tubing sizes (0.8-3.2 mm ID), with a standard deviation of less than 0.3 million cells. Similar results were obtained with the smaller tubings. Accordingly, this evidence indicates that the pumping velocity and cell concentration have no substantial effect on HDF cell viability when pumped through 0.8 mm to 3.2 mm ID tubing during time periods typical for bag filling operations according to the invention.

### Example 5

### Effect of Source Container Agitation on Dispensed Cell Concentration

A consistent concentration of dispensed cells in each product dose is a vital aspect of providing a safe and reliable product. The duration of the filling process for a complete product batch may allow settling of cells in the source and disparity in cell concentration from the beginning to the end of the run. The inventors have therefore investigated whether agitation the source bag throughout the run would provide a more constant cell concentration in the serially filled bags.

### Materials and Equipment

Chinese Hamster Ovary cells (CHO)

Flexicon Pump—model DF6

Orbital Shaker—model E2

Nucleocounter®

NucleoCassettes®

NucleoView® software

Ring stand

Cell Source Bag—P/N 84-711-032

2 L Nalgene Bottle—P/N 04443

Centrifuge tubes—various

These tests used CHO cells at a cell density of 1.6 million cells/mL in a 1.6 L of liquid culture medium (PowerCHO-2®, Lonza) The pump was configured with volume=10 mL, acceleration=100 rpm, velocity=400 rpm, reverse=1.0 mL, and delay=25 sec. The flexible cell source bag was a Configuration B 2 L bag provided by Flexicon Corporation. The collection container was a 2 L Nalgene bottle. Samples were collected at 0, 5, 10, 15, 20, 30, 40, 50 and 60 minutes, in 15 mL centrifuge tubes, and were tested for cell concentration and % viability as in the previous examples. Each test was run for one hour to simulate a full production run. A control run was done with no agitation of the source bag. The test run used a source bag that was mechanically agitated by connection to a ring stand attached to an orbital shaker set to 100 rpm. The ring stand was clamped to a pole attached horizontally near the base of the bag to provide a prodding action during the motion of the ring stand. Prior to each run, the source bag was inverted to thoroughly suspend the cells. The source bag was completely drained after each test and the contents were combined with untested samples for reuse in the next test run. The starting cell concentration in each test ranged from 1.64 million cells/mL to 1.54±0.04 million cell/mL.

As shown in FIG. 8, the control run with no bag agitation consistent maintained the initial cell concentration for about 30 minutes, whereas three consecutive runs with (“rotary”) agitation of the source bag showed no decline in cell concentration through the entire one hour run. These results indicate that maintaining a uniform suspension of source cells during the filling process of the invention may be readily accomplished by mechanical agitation of the source suspension. This example supports the an embodiment of the invention comprising a sterile closed system from the source bag (that must be agitated to support consistent product filling over filling times >30 minutes) to the final product bag, with inline vacuum and purge sources.

### Example 6

### Effect of DMSO on Cell Viability During Pumping

Since exposure to dimethylsulfoxide (DMSO) can reduce viability of cells, the inventors have investigated whether DMSO makes cells more susceptible to shear during pumping according to the bag-filling process of the invention

### Materials and Equipment

Flexicon Pump—DF6

Nucleocounter and accessories

Conical tubes—various sizes

Human Dermal Fibroblasts (HDF)

DMSO—P/N 07198

Media—Plasmalyte A (P/N 07204)+5% Human Serum Antibody (P/N 07489)

The Flexicon pump was setup using the settings: volume=10 mL, acceleration=100 rpm, velocity=400 rpm, reverse=1.0 mL, and delay=25 sec. HDF cells were suspended in medium at 10 million cells/mL, and DMSO was added to a final concentration of 10%. 10 mL of cell suspension was dispensed by pumping at 20 sec intervals. A baseline sample was taken prior to dispensing and 10 samples were recorded. The samples were ‘fixed’ in Nucleocassettes as rapidly as possible with the first being finished at 3 minutes post addition of DMSO and the last being finished at 18 minutes post addition. The samples were analyzed during this process with the last one being finished within 30 minutes. These tests revealed an average change in % viability of 1.17±0.86% for the ten test samples, compared to the control that was not pumped. Hence, these results show no significant affect of DMSO upon viability of HDF cells during processing using automated pumps and sterile closed tubing system when processed in a timely manner according to the bag-filling process of the invention.

### Example 7

### Complete System Testing

With all of the components optimized, a large scale filling run was performed using bone marrow derived progenitor cells formulated at 10 million cells/mL in Plasmalyte® containing 5% HSA and 10% DMSO. Over 4 billion cells were formulated in about 400 mL and transferred to a 1 L bag (the “source bag”) for dispensing. The source bag was steriley connected to a pre-assembled tubing set in the configuration of FIG. 1, and 20 bags were filled sequentially with 18 mL of product using the Flexicon pumping system previously described. To mimic a much larger run, a 30 minute time lag was introduced after bag 10 was filled, and then bags 11-20 were filled after the 30 minute waiting period. All bags were filled within 70 minutes of formulation, and cryopreserved using a controlled rate freezer. Bag fill volume was measured by weighing the bags post fill, and after thawing by measuring total volume removed with a syringe. Guave Viacount® was utilized to quantify cell concentration and cell viability, and this was compared to the source bag pre-freeze to calculate total viable cell recovery.

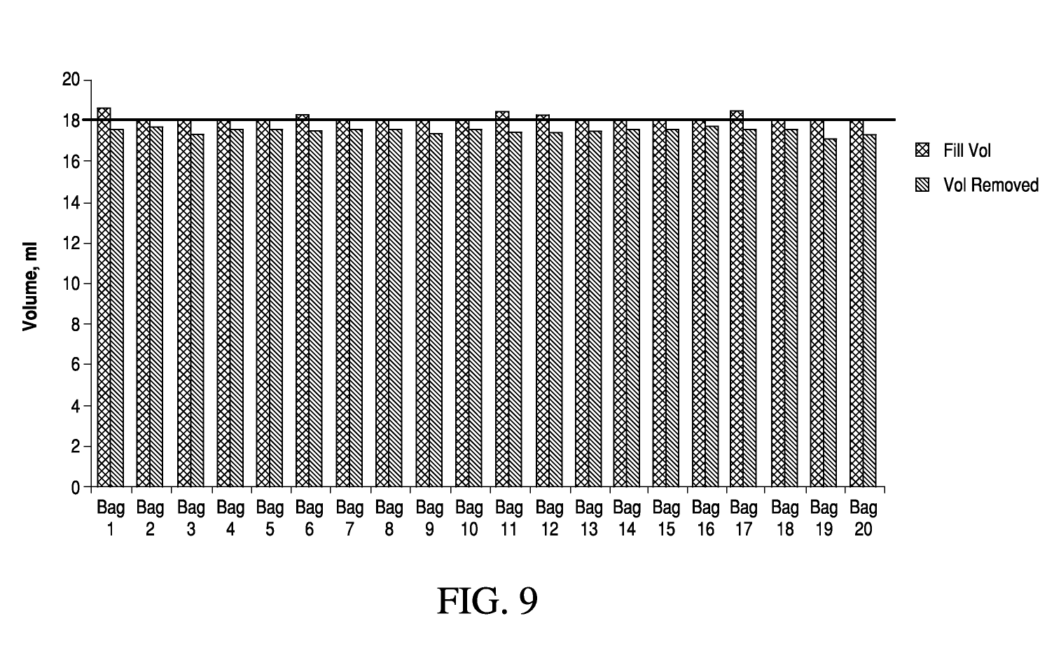

As shown in FIG. 9, total fill volume was accurate and precise over all 20 bags. Fill volume pre-freeze was 18.24±0.13 mL, and had a coefficient of variation (CV) of 0.69%. Total volume removed after thawing was 17.49±0.17 mL, with a CV of 0.94%. The target fill was 180 million cells/bag, and the total viable cell count average post thaw was 167.6 million±7.2 million cells (CV=4.3%), with a viability of 91.7%±2.5%. Total cell count per bag averaged 183.6 million cells/bag. This experiment demonstrates that this automatable process using a sterile closed system, connecting a source bag and final product bag(s) with in-line vacuum and purge sources for the filling of final product bags, is: 1) robust capacity of both volume and cell concentration, 2) successful in combining processing steps at different times, and adding an automatable purge step for minimizing product loss.

It will be understood that the embodiments of the present invention which have been described are illustrative of some of the applications of the principles of the present invention. Numerous modifications may be made by those skilled in the art without departing from the true spirit and scope of the invention.

What is claimed is:

1. A method for aseptically dispensing live mammalian cells into sterile, flexible bags in a non-sterile atmosphere, said method comprising the following steps:
(a) providing a plurality of said sterile flexible bags;
(b) providing said live mammalian cells suspended in a liquid contained in a sterile cell source container;
(c) connecting said sterile bags to said cell source container, a vacuum source and a sterile purging gas source to form a sterile system that is closed to the external environment, wherein said system selectively allows fluid flow between said sterile bags and said vacuum source or said cell source container or said sterile gas source;
(d) evacuating air from at least one of said flexible bags by selectively allowing fluid flow between said at least one bag and said vacuum source;
(e) dispensing a desired volume of said liquid for said at least one bag by selectively allowing said desired volume of liquid to flow between said at least one bag and said cell source container; and
(f) forcing into said at least one bag any of said desired volume of said liquid remaining in said system between said cell source container and said at least one bag by selectively allowing said sterile purging gas to flow from said gas container through said system to said at least one bag.

2. A method of claim 1 wherein said plurality of flexible bags, said cell source container, said vacuum source and said sterile purging gas source are fluidly connected together at least in part by flexible tubing and wherein at least one pinch valve that externally engages said flexible tubing is provided to selectively allow fluid flow between said sterile bags and said cell source container or said vacuum source or said gas source.

3. A method of claim 2 wherein a peristaltic pump unit is used to pump said liquid from said source container through said flexible tubing into said flexible bags.

4. The method of claim 3 wherein at least one of said pump unit and said valve is remotely operable by a controller.

5. The method of claim 2 wherein said flexible tubing comprises a main line and at least one branch line, wherein:
said main line is a length of flexible tubing having one end connected to said container and said gas source so as to selectively allow fluid flow into said main line from said container or said gas source;
each of said flexible bags is fluidly connected to said main line by a branch line, each of said branch lines being a length of flexible tubing having one end fluidly connected to one of said bags and the other end fluidly connected to said main line so as to allow said liquid or said gas to flow from said main line into said bag; and
said vacuum source is fluidly connected to said main line so as to allow said vacuum source to withdraw gas from each of said bags through said mainline and said branch lines.

6. A method of claim 2 further comprising sterilely sealing and disconnecting the branch line of a bag that has been filled.

7. A method of claim 1 wherein viability of cells in the last bag to be filled is at least 90% of the initial viability of cells in said cell source container before dispensing any of said liquid to fill said bags.

8. The method of claim 3 wherein the flow rate of said liquid through said tubing is from about 10 mL/min to about 1000 mL/min, density of cells in suspension is from about 1 to about 30 million/mL and said liquid comprises from 0 to about 10% dimethylsulfoxide.

9. A method of storing live mammalian cells comprising dispensing said cells into sterile, flexible bags according to the method of claim 6 and, after disconnecting a bag that has been filled, cryogenically preserving said cells in said bag.

10. An apparatus for aseptically dispensing live mammalian cells into sterile, flexible bags in a non-sterile atmosphere, said apparatus comprising:
a plurality of said sterile flexible bags;
a cell source container configured to contain said live mammalian cells suspended in a liquid;
a vacuum source; and
a sterile purging gas source
wherein said plurality of flexible bags, said cell source container, said vacuum source and said gas source are fluidly connected together to form a sterile system that is closed to the external environment such that said system selectively allows fluid flow between said sterile bags and said cell source container or said vacuum source or said gas source.

11. The apparatus of claim 10 wherein said plurality of flexible bags, said cell source container, said vacuum source and said sterile purging gas source are fluidly connected together at least in part by flexible tubing and wherein at least one pinch valve that externally engages said flexible tubing is provided to selectively allow fluid flow between said sterile bags and said cell source container or said vacuum source or said gas source.

12. An apparatus of claim 11 further comprising a peristaltic pump unit configured to pump said liquid from said source container through said flexible tubing into said flexible bags.

13. The apparatus of claim 12 wherein at least one of said pump unit and said pinch valve is remotely operable by a controller.

14. The apparatus of claim 12 wherein said flexible tubing comprises a main line and at least one branch line, wherein:
said main line is a length of flexible tubing having one end connected to said container and said gas source so as to selectively allow fluid flow into said main line from said container or said gas source;
each of said flexible bags is fluidly connected to said main line by a branch line, each of said branch lines being a length of flexible tubing having one end fluidly connected to one of said bags and the other end fluidly connected to said main line so as to allow said liquid or said gas to flow from said main line into said bag; and
said vacuum source is fluidly connected to said main line so as to allow said vacuum source to withdraw gas from each of said bags through said mainline and said branch lines.
